(19) United States
(12) Patent Application Publication
(10) Pub. No.: US 2022/0107513 A1
(43) Pub. Date: Apr. 7, 2022

(54) HIGH VISIBILITY SAFETY EYEWEAR ASSEMBLY

(71) Applicant: George A. Smith, III, Durham, NC (US)

(72) Inventor: George A. Smith, III, Durham, NC (US)

(21) Appl. No.: 17/061,649

(22) Filed: Oct. 2, 2020

(51) Int. Cl. G02C 11/04; G02C 11/00; A61F 9/02

(52) U.S. Cl. CPC G02C 11/04; A61F 9/029; G02C 11/10

(57) ABSTRACT

A high visibility safety eyewear assembly includes a frame and a pair of temples that are attached to and extend rearwardly of the frame. A lens is attached to and extends downwardly from the frame. The lens includes lateral sides extending to a forward section of each of the temples. The lens are comprised of an impact resistant material. A reflective material is attached to and extends along a front side of the frame and further extends beyond the frame and onto each of the temples. A light emitter is mounted on a first temple of the pair of temples. The light emitter is directed forward of the frame. An actuator is engaged with the light emitter to turn the light emitter on or off.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to safety eyewear device and more particularly pertains to a new safety eyewear device for providing high visibility of a person wearing safety glasses.

### (2) Description of Related Art Including Information Disclosed Under 37 CFR 1.97 and 1.98

The prior art relates to safety eyewear devices that are used, typically, for protecting a person's eyes from objects that might otherwise strike their eyes.

### BRIEF SUMMARY OF THE INVENTION

An embodiment of the disclosure meets the needs presented above by generally comprising a frame and a pair of temples that are attached to and extend rearwardly of the frame. A lens is attached to and extends downwardly from the frame. The lens includes lateral sides extending to a forward section of each of the temples. The lens are comprised of an impact resistant material. A reflective material is attached to and extends along a front side of the frame and further extends beyond the frame and onto each of the temples. A light emitter is mounted on a first temple of the pair of temples. The light emitter is directed forward of the frame. An actuator is engaged with the light emitter to turn the light emitter on or off.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front isometric view of a high visibility safety eyewear assembly according to an embodiment of the disclosure.

FIG. 2 is a left side view of an embodiment of the disclosure.

FIG. 3 is a right side view of an embodiment of the disclosure.

FIG. 4 is a front view of an embodiment of the disclosure.

FIG. 5 is a bottom view of an embodiment of the disclosure.

FIG. 6 is a side view of a control and satchel of an embodiment of the disclosure.

FIG. 7 is a side in-use view of an embodiment of the disclosure.

FIG. 8 is a schematic view of an embodiment of the disclosure.

### DETAILED DESCRIPTION OF THE INVENTION

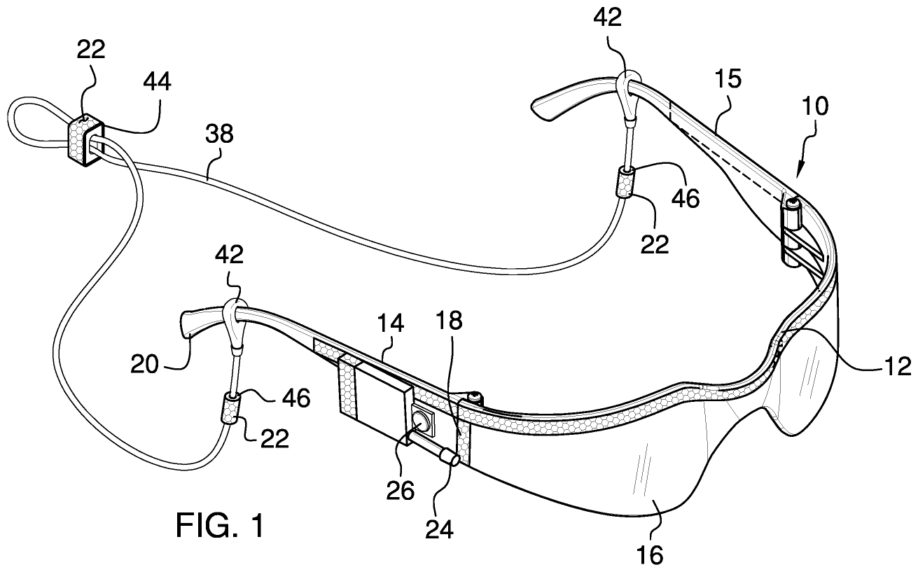

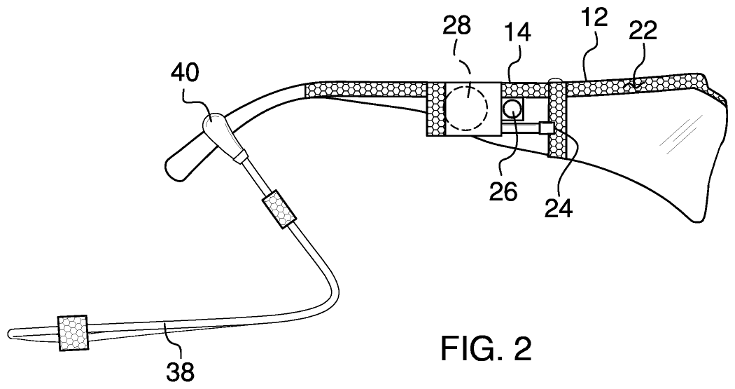

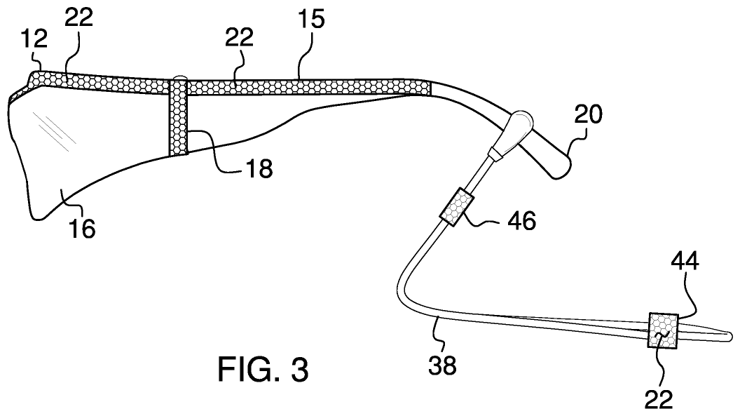

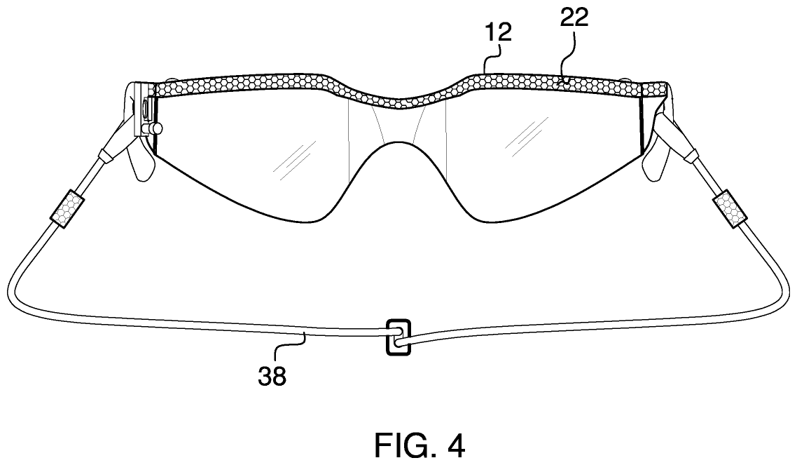

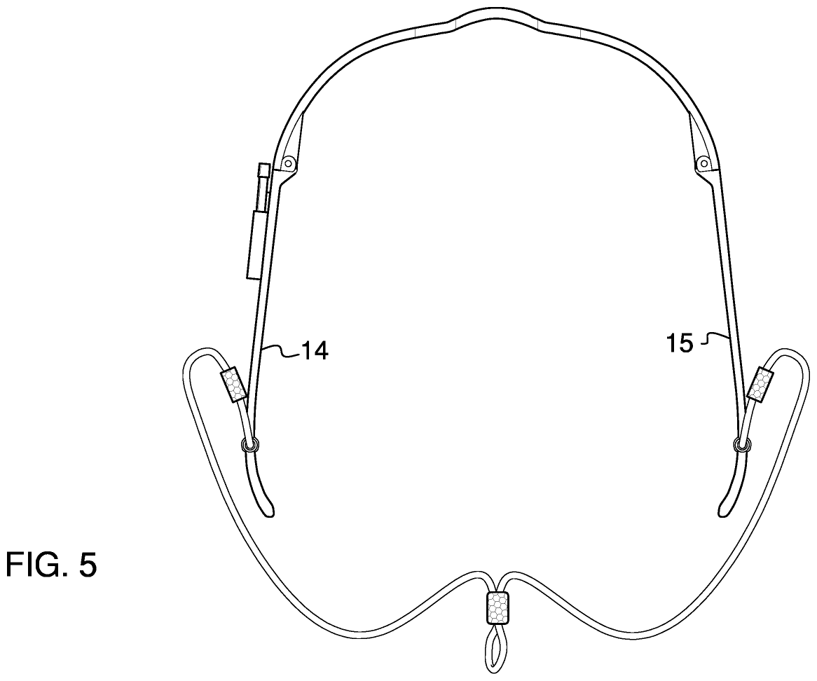

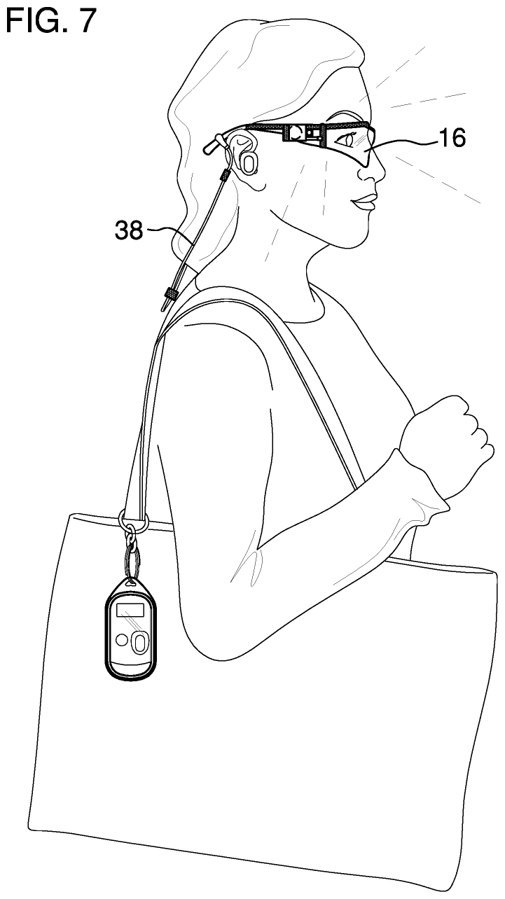

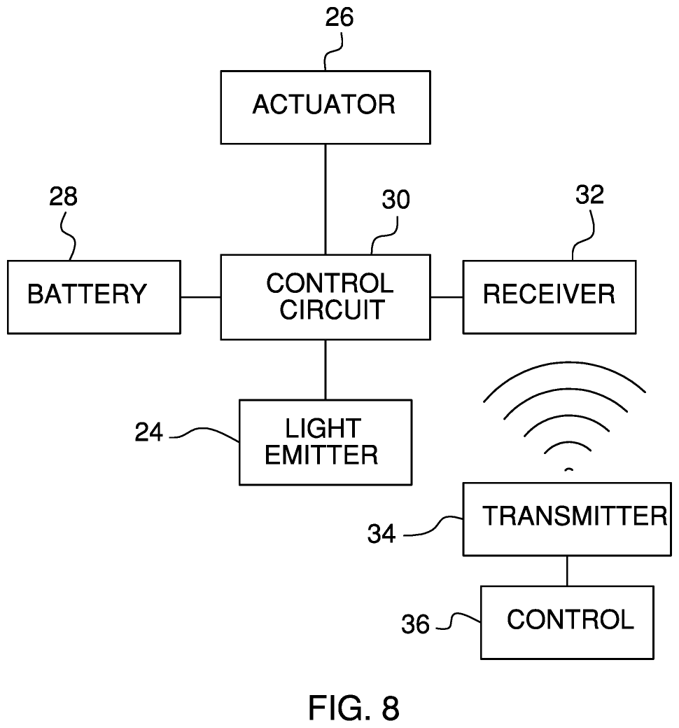

With reference now to the drawings, and in particular to FIGS. 1 through 8 thereof, a new safety eyewear device embodying the principles and concepts of an embodiment of the disclosure and generally designated by the reference numeral 10 will be described.

As best illustrated in FIGS. 1 through 8, the high visibility safety eyewear assembly 10 generally comprises a frame 12 and a pair of temples 14, 15 is attached to and extends rearwardly of the frame 12. The frame 12 may include a bridge and nose pads or may be a single piece of material extending across a top of the assembly 10 as shown in the Figures. A lens 16 is attached to and extends downwardly from the frame 12. The lens 16 includes lateral sides extending toward a forward section 18 of each of the temples 14, 15. A rear section 20 of the temples 14, 15 extends downwardly arcuately to engage a person's ears. The lens 16 is comprised of an impact resistant material which is conventional to safety glasses. The lens 16 is substantially transparent and may be tinted, colored, or clear.

A reflective material 22 is attached to and extends along a front side of the frame 12. The reflective material 22 extends beyond the frame 12 and onto each of the temples 14. As can be seen in the figures, the frame 12 may be curved and the temples 14, 15 attached to the frame 12 by hinges 24. The reflective material 22 extends beyond the junctures of the frame 12 and the temples 14, 15 and onto the temples 14, 15. The reflective material 22 may further extend down along an edge of the lens 16 where the frame 12 meets the temples 14.

A light emitter 24 is mounted on a first temple 14 of the pair of temples 14, 16. The light emitter 24 is directed forward of the frame 12 and will typically comprise a light emitting diode. An actuator 26 is engaged with the light emitter 24 to turn the light emitter 24 on or off. The actuator 26 is mounted on the first temple 14 and is located adjacent to the light emitter 24. A battery 28 may be mounted in a housing on the first temple 14 for powering the light emitter 24.

A control circuit 30 is electrically coupled to the actuator 26 and the light emitter 24. The control circuit 30 controls light emission of the light emitter 24 wherein the light emission of the light emitter 24 is selectable between continuous light emission and variable speed flashes of light emission by actuation of the actuator 26.

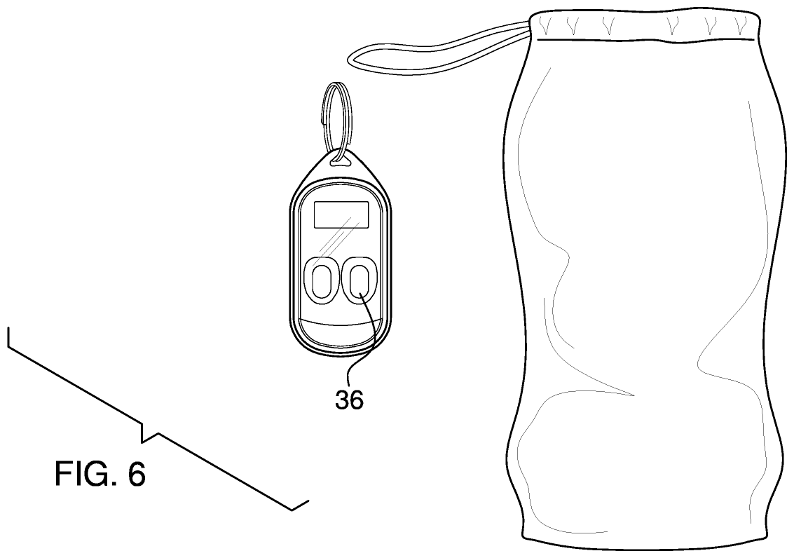

A receiver 32 is electrically coupled to the control circuit 30. A transmitter 34 is in wireless communication with the receiver 32. A control 36 is electrically coupled to the transmitter 34 and is actuated to control the light emitter 24. The control 36 may comprise a dedicated remote control such as shown in FIG. 6. Alternatively, the control 36 may include a cellular phone programed with an application for connection to the receiver 32 and thereafter actuate the light emitter 24 as desired.

A lanyard 38 has a first end 40 and a second end 42. The first end 40 is attached to one of the temples 14 and the second end 42 is attached to another one of the temples 15. The lanyard 38 is used typically to retain the frame 12 on the wearer's head. A stop 44, used for tightening the lanyard 38, is attached to the lanyard 38 between the first 40 and second 42 ends. The stop 44 has the reflective material 22 positioned thereon. Additional sleeves 46 may be positioned on the lanyard 38 and again may include reflective material 22.

In use, assembly 10 is worn in a conventional manner to protect a person's eyes. However, the reflective material utilized on the frame 12 and temples 14, 15, and the light emitter 24, ensure that the wearer of the assembly 10 is highly visible to, such as for example, oncoming traffic.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A protective eyewear assembly comprising:
a frame and a pair of temples being attached to and extending rearwardly of said frame;
a lens being attached to and extending downwardly from said frame, said lens including lateral sides extending to a forward section of each of said temples, said lens being comprised of an impact resistant material;
a reflective material being attached to and extending along a front side of said frame, said reflective material extending beyond said frame and onto each of said temples;
a light emitter being mounted on a first temple of said pair of temples, said light emitter being directed forward of said frame; and
an actuator being engaged with said light emitter to turn said light emitter on or off.

2. The protective eyewear assembly according to claim 1, further including a control circuit being electrically coupled to said actuator and said light emitter, said control circuit controlling light emission of said light emitter wherein the light emission of said light emitter is selectable between continuous light emission and variable speed flashes of light emission by actuator of said actuator.

3. The protective eyewear assembly according to claim 2, further including:
a receiver being electrically coupled to said control circuit;
a transmitter being in wireless communication with said receiver; and
a control being electrically coupled to said transmitter, said control being actuated to control said light emitter.

4. The protective eyewear assembly according to claim 1, further including a lanyard having a first end and a second end, said first end being attached to one of said temples and said second end being attached to another one of said temples.

5. The protective eyewear assembly according to claim 4, further including a stop being attached to said lanyard between said first and second ends, said stop having a reflective material positioned thereon.

6. The protective eyewear assembly according to claim 3, further including a lanyard having a first end and a second end, said first end being attached to one of said temples and said second end being attached to another one of said temples.

7. A protective eyewear assembly comprising:
a frame and a pair of temples being attached to and extending rearwardly of said frame;
a lens being attached to and extending downwardly from said frame, said lens including lateral sides extending to a forward section of each of said temples, said lens being comprised of an impact resistant material;
a reflective material being attached to and extending along a front side of said frame, said reflective material extending beyond said frame and onto each of said temples;
a light emitter being mounted on a first temple of said pair of temples, said light emitter being directed forward of said frame;
an actuator being engaged with said light emitter to turn said light emitter on or off;
a control circuit being electrically coupled to said actuator and said light emitter, said control circuit controlling light emission of said light emitter wherein the light emission of said light emitter is selectable between continuous light emission and variable speed flashes of light emission by actuator of said actuator;
a receiver being electrically coupled to said control circuit;
a transmitter being in wireless communication with said receiver;
a control being electrically coupled to said transmitter, said control being actuated to control said light emitter;
a lanyard having a first end and a second end, said first end being attached to one of said temples and said second end being attached to another one of said temples; and
a stop being attached to said lanyard between said first and second ends, said stop having a reflective material positioned thereon.
